(19) United States
(12) Patent Application Publication
(10) Pub. No.: US 2025/0275913 A1
(43) Pub. Date: Sep. 4, 2025

(54) HAIR, SCALP AND SKIN TREATMENT COMPOSITIONS

(71) Applicant: ROZ Hair Inc., La Cañada, CA (US)

(72) Inventor: Mara Roszak, La Cañada, CA (US)

(21) Appl. No.: 19/069,066

(22) Filed: Mar. 3, 2025

Related U.S. Application Data
(60) Provisional application No. 63/560,323, filed on Mar. 1, 2024.

(51) Int. Cl. A61K 8/9789; A61K 8/9794; A61Q 5/00; A61Q 5/02; A61Q 5/06; A61Q 5/12; A61Q 19/00

(52) U.S. Cl. CPC A61K 8/9789; A61K 8/9794; A61Q 5/002; A61Q 5/004; A61Q 5/02; A61Q 5/06; A61Q 5/12; A61Q 19/00

(57) ABSTRACT

Provided herein are hair, scalp, and skin treatment compositions that comprise natural ingredients.

### CROSS-REFERENCE TO RELATED APPLICATION

This application claims the benefit of U.S. Provisional Patent Application No. 63/560,323, filed Mar. 1, 2024, the entire disclosure of which is incorporated herein by reference.

### FIELD

This disclosure relates to cosmetic, hair, scalp and skin treatment compositions that comprise natural ingredients.

### BACKGROUND

Many consumers who style their hair need products that reduce hair breakage or hair dryness or scalp itchiness. Many existing products use harsh chemicals which aggravate hair, scalp and skin problems leaving hair undesirably dry, damaged, frizzy, and unmanageable.

There is a continuing need for improved hair, scalp, and skin conditioning compositions, particularly compositions that are gentle on the hair, scalp and/or skin.

### SUMMARY

Provided herein are compositions primarily formulated of natural ingredients and capable of providing hair, scalp and skin treatments without substantial damage to the hair, scalp or skin. The compositions may comprise ginger root extract, grape extract, walnut extract, or frankincense. The compositions herein may be in the form of a shampoo, a dry shampoo, a conditioner, a dry conditioner, a lotion, a cream, a spray, a powder, a serum, a milk serum, a gel, a mask, a spray, a mist, a balm, a foam, a wax, an exfoliant, a scrub, or a combination of forms.

### BRIEF DESCRIPTION OF THE DRAWINGS

FIG. 1 is an untreated hair fiber image at 2000× magnification obtained by SEM.

FIG. 2 is a treated hair fiber image at 2000× magnification obtained by SEM.

FIG. 3 is a plot of the number of broken hair fibers in an untreated control group and a hair mask composition treatment group.

### DETAILED DESCRIPTION

Typical hair and scalp conditioning products leave a product residue on hair or scalp which builds up after repeated uses of the product. The same is true of many cosmetics and other personal care products for use on hair, scalp or skin. The product build up leads to dry hair, dry or itchy skin, dry or itchy scalp, hair breakage, and/or hair loss. Sometimes, typical hair and scalp conditioning products affect the color applied to hair, on the head or other areas of the body having hair, thereby causing hair color to look dull and lifeless

It has been found that a combination of natural anti-inflammatory compounds, natural emollients, natural oils, and natural ingredients can improve blood circulation in the scalp and skin and can alleviate problems associated with typical hair, scalp, and skin conditioning products.

As used herein “natural ingredients” refers to ingredients found in nature, e.g., nut oils, plant resins/gums, extracts from part of plants such as roots, fruits, seeds, bark, or leaves of plants where the extraction process leaves the chemical structure of the extracted ingredient substantially similar to the chemical structure of the ingredient in the plant. Non-limiting examples of natural ingredients include e.g., bees wax, coconut alkenes, etc.

As used herein % w/w means percent weight of the specified ingredient in the total weight of the composition.

In some embodiments, compositions herein are hair, scalp and skin conditioning compositions. In some embodiments, the compositions described herein may be in the form of a shampoo, a dry shampoo, a conditioner, a dry conditioner, a lotion, a cream, a spray, a powder, a serum, a milk serum, a gel, a mask, or a combination of forms. In other embodiments, the compositions herein may be in the form of a mist, a balm, a foam, a wax, waxes, an exfoliant, a scrub, an exfoliating scrub, or a combination of any of these forms. In some embodiments, the compositions described herein may be oil-based formulations. In other embodiment, the compositions herein may be included in capsules, softgels, caplets, tablets, tabs, pills, powders, or any other appropriate delivery form, or a combination of any of these forms.

Provided herein are compositions primarily formulated of natural ingredients and capable of providing hair, scalp, or skin treatments without any substantial damage to the hair, scalp, or skin. The natural ingredients can have soothing, hydrating, and/or nourishing properties. The compositions described herein can be vegan, organic, sustainably sourced, and/or cruelty free. In some embodiments, the “primarily formulated” means the composition comprises at least 50% w/w, at least 60% w/w, at least 70% w/w, at least 80% w/w, or at least 90% w/w of natural ingredients and/or water.

In some embodiments, the compositions described herein provide barrier protection for the hair, scalp, and skin. In such embodiments, the compositions herein create a hydrophobic (water-repelling) layer on the hair surface. A more hydrophobic surface can protect hair from environmental damage and water-related color loss. Some embodiments of the compositions provide melanin protection in sunlight, thereby providing color protection. Other embodiments of the compositions described herein can provide a reduction in color fading in dyed hair after treatments.

Some embodiments include compositions that provide customized hair, scalp, and skin health based on seasonal variations in temperature and humidity. In such embodiments, hair, scalp and skin health is protected in different environments to maintain a healthy hair ecosystem year-round.

In other embodiments, hair, scalp, and skin repair may be achieved by the compositions herein via the repair of disulfide bonds. Disulfide bonds form between two cysteine amino acid moieties, cross-linking keratin molecules together. These bonds stabilize and hold keratin molecules together, and can influence hair shape, strength, and texture (e.g., curliness or straightness, and overall elasticity of hair). In such embodiments, mechanical repair of hair can be achieved, resulting in improved tensile strength.

Other embodiments include compositions that can reduce static electricity in the hair and scalp. In such embodiments, the compositions can reduce hair frizz.

In some embodiments, the compositions described herein can be useful for cellular detoxification, thereby improving follicle health in the scalp. In other embodiments, the compositions described herein can be useful for detoxification of hair. In some embodiments, environmental pollutants and toxins can be removed at the cellular level, reducing oxidative stress to the hair and scalp. In some of these embodiments, reduction in levels of malondialdehyde (MDA) can result in detoxification. MDA is a byproduct of lipid peroxidation and an indirect indicator of oxidative stress. In other embodiments, the compositions herein can reduce Reactive Oxygen Species (ROS) in hair follicles or the scalp or skin. In still another embodiment, the compositions of the present disclosure can improve levels of antioxidant enzyme activity. Some compositions can provide protection for antioxidant enzymes such as superoxide dismutase (SOD), catalase, and glutathione peroxidase in cells of the scalp, skin, or hair.

In some embodiments, hair follicle DNA protection can be provided by the compositions described herein. In such cases, protection against UVA rays, UVB rays, sunlight, and environmental metals can be achieved.

In other embodiments, the compositions of the present disclosure can protect, enhance, or repair hair growth phases. In such embodiments, operation of the anagen phase, the catagen phase, and/or the telogen phase can be protected, enhanced and/or repaired.

In some embodiments, the compositions herein comprise ginger root extract, grape extract, walnut extract, and frankincense. These natural components are discussed in more detail below.

In some embodiments, the shampoos herein comprise ginger root extract, grape extract, walnut extract, and frankincense.

In some embodiments, the dry shampoos herein comprise ginger root extract, grape extract, walnut extract, and frankincense.

In some embodiments, the conditioners herein comprise ginger root extract, grape extract, walnut extract, and frankincense.

In some embodiments, the dry conditioners herein comprise ginger root extract, grape extract, walnut extract, and frankincense.

In some embodiments, the lotions herein comprise ginger root extract, grape extract, walnut extract, and frankincense.

In some embodiments, the creams herein comprise ginger root extract, grape extract, walnut extract, and frankincense.

In some embodiments, the sprays herein comprise ginger root extract, grape extract, walnut extract, and frankincense.

In some embodiments, the powders herein comprise ginger root extract, grape extract, walnut extract, and frankincense.

In some embodiments, the serums herein comprise ginger root extract, grape extract, walnut extract, and frankincense.

In some embodiments, the milk serums herein comprise ginger root extract, grape extract, walnut extract, and frankincense.

In some embodiments, the gels herein comprise ginger root extract, grape extract, walnut extract, and frankincense.

In some embodiments, the masks herein comprise ginger root extract, grape extract, walnut extract, and frankincense.

In some embodiments, the mists herein comprise ginger root extract, grape extract, walnut extract, and frankincense. a lotion a spray,

In some embodiments, the balms herein comprise ginger root extract, grape extract, walnut extract, and frankincense

In some embodiments, the foams herein comprise ginger root extract, grape extract, walnut extract, and frankincense.

In some embodiments, the waxes herein comprise ginger root extract, grape extract, walnut extract, and frankincense.

In some embodiments, the exfoliants herein comprise ginger root extract, grape extract, walnut extract, and frankincense.

In some embodiments, the scrubs herein comprise ginger root extract, grape extract, walnut extract, and frankincense.

In some embodiments, the exfoliating scrubs herein comprise ginger root extract, grape extract, walnut extract, and frankincense.

Ginger root extract, also referred to as Zingiber officinale root extract, is a natural anti-inflammatory substance comprising vitamins and minerals which can soothe skin and scalp irritation and itchiness. Ginger root extract can also strengthen hair from the root, promoting hair resilience and reducing breakage for healthier looking hair. In some embodiments, ginger root extract can also be referred to a ginger or ginger extract.

Grape extract, also referred to as Vitis vinifera fruit extract and/or Vitis vinifera Vine Sap, is rich in antioxidants like resveratrol and vitamin E. Grape extract can preserve hair color and protects hair from breakage. Grape extract can help protect the hair, scalp and skin from environmental stressors, improving texture and promoting a balanced, healthy scalp.

Walnut extract, also referred to as hydrolyzed walnut extract and/or hydrolyzed Juglans regia extract, is rich in essential omega-3 fatty acids. Hydrolyzed walnut extract can nourish the hair and help preserve color, preventing fading while maintaining a smooth, glossy finish. Walnut extract can also strengthen hair strands and protect hair from damage.

Frankincense, also referred to as Boswellia Serrata resin extract, is deeply nourishing, and can help hydrate the hair. Frankincense can restore moisture balance and enhances shine for a healthy, vibrant look. Frankincense also can improve blood circulation in the skin and scalp and can restore the skin and scalp or promote skin and scalp restoration.

In some embodiments, the compositions described herein comprise about 0.01% to about 3% (w/w) of ginger root extract. In other embodiments, the compositions comprise about 0.01% to about 0.025%, about 0.025% to about 0.05%, about 0.05% to about 0.075%, about 0.075% to about 0.1%, about 0.1% to about 0.2%, about 0.2% to about 0.3%, about 0.3% to about 0.4%, about 0.4% to about 0.5%, about 0.5% to about 0.6%, about 0.6% to about 0.7%, about 0.7% to about 0.8%, about 0.9% to about 1%, about 1% to about 1.2%, about 1.2% to about 1.4%, about 1.4% to about 1.6%, about 1.6% to about 1.8%, about 1.8% to about 2%, about 2% to about 2.2%, about 2.2% to about 2.4%, about 2.4% to about 2.6%, about 2.6% to about 2.8%, about 2.8% to about 3%, or about 0.01%, about 0.05%, about 0.1%, about 0.5%, about 1%, about 1.5%, about 2%, about 2.5%, about 3%, or any weight percentage of ginger root extract in a range bounded by any of these values. In some embodiments, the compositions described herein comprise at least 3% (w/w) of ginger root extract.

In some embodiments, the compositions of the present disclosure comprise about 0.01% to about 3% (w/w) of grape extract. In other embodiments, the compositions comprise about 0.01% to about 0.025%, about 0.025% to about 0.05%, about 0.05% to about 0.075%, about 0.075% to about 0.1%, about 0.1% to about 0.2%, about 0.2% to about 0.3%, about 0.3% to about 0.4%, about 0.4% to about 0.5%, about 0.5% to about 0.6%, about 0.6% to about 0.7%, about 0.7% to about 0.8%, about 0.9% to about 1%, about 1% to about 1.2%, about 1.2% to about 1.4%, about 1.4% to about 1.6%, about 1.6% to about 1.8%, about 1.8% to about 2%, about 2% to about 2.2%, about 2.2% to about 2.4%, about 2.4% to about 2.6%, about 2.6% to about 2.8%, about 2.8% to about 3%, or about 0.01%, about 0.05%, about 0.1%, about 0.5%, about 1%, about 1.5%, about 2%, about 2.5%, about 3%, or any weight percentage of grape extract in a range bounded by any of these values. In some embodiments, the compositions of the present disclosure comprise at least 3% (w/w) of grape extract.

In other embodiments, the compositions of the present disclosure comprise about 0.01% to about 3% (w/w) hydrolyzed walnut extract. In other embodiments, the compositions comprise about 0.01% to about 0.025%, about 0.025% to about 0.05%, about 0.05% to about 0.075%, about 0.075% to about 0.1%, about 0.1% to about 0.2%, about 0.2% to about 0.3%, about 0.3% to about 0.4%, about 0.4% to about 0.5%, about 0.5% to about 0.6%, about 0.6% to about 0.7%, about 0.7% to about 0.8%, about 0.9% to about 1%, about 1% to about 1.2%, about 1.2% to about 1.4%, about 1.4% to about 1.6%, about 1.6% to about 1.8%, about 1.8% to about 2%, about 2% to about 2.2%, about 2.2% to about 2.4%, about 2.4% to about 2.6%, about 2.6% to about 2.8%, about 2.8% to about 3%, or about 0.01%, about 0.05%, about 0.1%, about 0.5%, about 1%, about 1.5%, about 2%, about 2.5%, about 3%, or any weight percentage hydrolyzed walnut extract in a range bounded by any of these values. In other embodiments, the compositions of the present disclosure comprise at least 3% (w/w) hydrolyzed walnut extract.

In some embodiments, the compositions of the present disclosure comprise about 0.01% to about 3% (w/w) of frankincense. In other embodiments, the compositions comprise about 0.01% to about 0.025%, about 0.025% to about 0.05%, about 0.05% to about 0.075%, about 0.075% to about 0.1%, about 0.1% to about 0.2%, about 0.2% to about 0.3%, about 0.3% to about 0.4%, about 0.4% to about 0.5%, about 0.5% to about 0.6%, about 0.6% to about 0.7%, about 0.7% to about 0.8%, about 0.9% to about 1%, about 1% to about 1.2%, about 1.2% to about 1.4%, about 1.4% to about 1.6%, about 1.6% to about 1.8%, about 1.8% to about 2%, about 2% to about 2.2%, about 2.2% to about 2.4%, about 2.4% to about 2.6%, about 2.6% to about 2.8%, about 2.8% to about 3%, or about 0.01%, about 0.05%, about 0.1%, about 0.5%, about 1%, about 1.5%, about 2%, about 2.5%, about 3%, or any weight percentage of frankincense in a range bounded by any of these values. In some embodiments, the compositions of the present disclosure comprise at least 3% (w/w) of frankincense.

In certain embodiments, the compositions herein further comprise at least one of the following additives: a thickener, a surfactant, a dye, a fragrance, and/or a preservative. In some embodiments, a natural thickener is used, e.g. a natural thickener can be, but is not limited to, beeswax or xanthan gum. In other embodiments, a natural surfactant is used, e.g., a natural surfactant can be, but is not limited to, lauryl glucoside. In some embodiments, a natural fragrance is used, e.g., a natural fragrance can be, but is not limited to, a citrus or floral extract. In some embodiments, a natural preservative is used, e.g., a natural preservative can be, but is not limited to, a citric acid or salt.

In other embodiments, the compositions herein can comprise one or more of the following additives: Cocos nucifera oil, behentrimonium chloride, caprylic/capric triglyceride, stearyl alcohol, polyquaternium-37, acrylates/stearyl methacrylate copolymer, cetrimonium chloride, dicaprylyl carbonate, Helianthus annuus (sunflower) extract, Rosmarinus officinalis leaf extract, hydroxypropyl starch phosphate, isopropyl alcohol, Oryza sativa bran extract, quaternium-87, sodium citrate, sorbitan oleate, tocopherol, panthenol, Furcellaria lumbricalis extract, Vaccinium myrtillus seed oil, Amaranthus caudatus seed extract, hydrolyzed chestnut extract, hydroxyacetophenone, Helianthus annuus seed oil, hydroxypropyltrimonium hyaluronate, sodium metabisulfite, tocopheryl acetate, bisabolol, diisostearyl malate, phenethyl alcohol, hydroxycitronellal, sodium cocoyl isethionate, lauryl glucoside, sodium C₁₄₋₁₆ olefin sulfonate, sodium lauroyl methyl isethionate, cocamidopropyl betaine, sodium methyl oleoyl taurate, coconut acid, coco-glucoside, cocamide mipa, sodium isethionate, 1,2-hexanediol, caprylyl glycol, sodium hydroxide, glutaral, glycerin, cetearyl alcohol, Persea gratissima (avocado) oil, behentrimonium methosulfate, cetyl alcohol, dicetyldimonium chloride, phenoxyethanol, ethylhexylglycerin, butylene glycol, guar hydroxypropyltrimonium chloride, propylene glycol, sodium gluconate, Pisum sativum (pea) peptide, cetyl hydroxyethylcellulose, limonene, geraniol, linalool, sorbitol, citric acid, lecithin, leuconostoc/radish root ferment filtrate, hydrated silica, sodium lauroyl lactylate, xanthan gum, disodium phosphate, silica dimethyl silylate, sodium benzoate, sodium phosphate, potassium sorbate, ceramide NP, ceramide AP, phytosphingosine, cholesterol, carbomer, ceramide EOP, fragrance (parfum), or any combination thereof. This list is not meant to be limiting in any way, and other suitable additives can be used in the compositions described herein.

In some embodiments, the compositions herein impart a silky wet feel to hair and inhibit fly-away up to about 35% relative humidity.

In some embodiments, the compositions herein are rinsed off after application to the hair, scalp, and/or skin.

In other embodiments, the compositions herein are applied to the hair, scalp, and/or skin and are not rinsed off.

In some embodiments, the compositions herein are applied on a daily basis. In other embodiments, the compositions herein are applied based on an individual's needs.

Some embodiments include compositions that are intended for daily use.

Other embodiments include compositions that are intended for weekly use.

In some embodiments, the compositions herein have substantially reduced product build up after repeated use.

In some embodiments, the compositions herein have a pH in the range of about 4 to about 6. In some embodiments, the compositions herein have a pH in the range of about 3 to about 7, about 4 to about 5, about 4.5 to about 7, about 4.5 to about 5, about 5 to about 6, about 5 to about 7, about 6 to about 7, or any pH in a range bounded by any of these values. In some embodiments, the compositions herein have a pH that is substantially close to a neutral pH of about 7.

Some embodiments include compositions that retain hair color and reduce breakage of hair strands. In some embodiments, a decrease in hair breakage of greater than about 10%, greater than about 20%, or greater than about 30% has been achieved using the compositions of the present disclosure.

In some embodiments, compositions herein are in the form of a shampoo, a dry shampoo, a conditioner, a dry conditioner, a lotion, a cream, a spray, a powder, a serum, a milk serum, a gel, a mask, a spray, a mist, a balm, a foam, a wax, an exfoliant, a scrub, a capsule, a softgel, or a combination of forms. In some embodiments, the combination of ginger root extract, grape extract, walnut extract, and frankincense comprise about 0.5% to about 5% w/w, about 0.5% to about 1%, about 1% to about 1.5%, about 1.5% to about 2%, about 2% to about 2.5%, about 2.5% to about 3%, about 3% to about 3.5%, about 3.5% to about 4%, about 4% to about 4.5%, about 4.5% to about 5%, about 0.5% to about 2%, about 1% to about 2%, about 2.5% to about 5%, or about 1%, about 2%, about 3%, about 4%, or any weight percentage in a range bounded by any of these ranges in the aforementioned composition forms.

In some embodiments, compositions herein are hair, scalp, and skin conditioning compositions. In some embodiments, the conditioning composition is a rinse-off conditioner. In some embodiments, the conditioning composition is a leave-in conditioner.

In some embodiments, compositions herein are aqueous. In this respect, the amount of water in the composition can be any suitable amount, e.g., from about 2% to about 98% by weight of the composition, from about 20% to about 90% by weight of the composition, from about 30% to about 80% by weight of the composition, or from about 40% to about 70% by weight of the compositions.

In some embodiments, the compositions herein can thoroughly cleanse the hair.

In some embodiments, the compositions herein does not cause hair color to fade.

In some embodiments, the compositions herein do not cause dandruff.

In some embodiments, the compositions herein can rinse out easily.

In some embodiments, the compositions herein can leave hair feeling clean.

In some embodiments, the compositions herein can leave no residue.

In some embodiments, the compositions herein do not cause the scalp and/or skin to feel dry or itchy.

In some embodiments, the compositions herein can leave hair, scalp, and/or skin feeling smooth.

In some embodiments, the compositions herein can produce a lather that is pleasing to a consumer.

In some embodiments, the compositions herein can leave the hair, scalp, and/or skin feeling clean.

In some embodiments, the compositions herein can thoroughly lather hair.

In some embodiments, the texture and consistency of the compositions herein can be pleasing to a user.

In some embodiments, the compositions herein can leave hair, scalp, and and/or looking healthy.

In some embodiments, the compositions herein can leave hair, scalp, and/or skin feeling hydrated.

In some embodiments, the compositions herein can leave hair, scalp, and skin feeling healthy.

In some embodiments, the compositions herein do not strip hair.

In some embodiments, the compositions herein can leave hair, scalp, and/or skin feeling free of buildup.

In some embodiments, the compositions herein can leave the hair, scalp, and/or skin feeling healthy.

In some embodiments, the compositions herein can leave hair feeling soft to the touch.

In some embodiments, the compositions herein can leave the scalp and/or skin feeling balanced.

In some embodiments, the compositions herein can leave the hair noticeably softer.

In some embodiments, the compositions herein can enhance the natural healthy texture of hair.

In some embodiments, the compositions herein can leave hair feeling conditioned.

In some embodiments, the compositions herein can cause hair to be instantly soft.

In some embodiments, the compositions herein can leave hair feeling nourished.

In some embodiments, the compositions herein can rinse out easily.

In some embodiments, the compositions herein can have a pleasing scent.

In some embodiments, the compositions herein can leave hair looking shiny.

In some embodiments, the compositions herein can detangle hair.

In some embodiments, the compositions herein can make hair easy to detangle after use.

In some embodiments, the compositions herein can cause hair to appear less damaged.

In some embodiments, the compositions herein do not leave hair feeling crunchy.

In some embodiments, the compositions herein can leave hair feeling smooth and soft to the touch.

In some embodiments, the compositions herein can feel lightweight on hair, scalp and/or skin.

In some embodiments, the compositions herein can leave hair feeling moisturized.

In some embodiments, the compositions herein can feel lightweight in hair.

In some embodiments, the compositions herein can leave hair, scalp and/or skin looking hydrated.

In some embodiments, the compositions herein can leave hair looking less frizzy.

In some embodiments, the compositions herein can easily tame frizz and fly-aways.

In some embodiments, the compositions herein can give hair, scalp, and/or skin a smooth, defined appearance.

In some embodiments, the compositions herein can leave hair looking smoother.

In some embodiments, the compositions herein can provide frizz control.

In some embodiments, the compositions herein can cause hair to be more manageable.

In some embodiments, the compositions herein can feel lighter weight in comparison to other styling products.

In some embodiments, the compositions herein can leave hair looking healthier.

In some embodiments, the compositions herein can easily defrizz hair.

In some embodiments, the compositions herein can leave hair feeling buildable and layerable.

In some embodiments, the compositions herein can make styling easier.

In some embodiments, the compositions herein can leave hair appearing less damaged.

In some embodiments, the compositions herein can provide long lasting frizz control.

In some embodiments, the compositions herein can detangle hair.

In some embodiments, the compositions herein can leave the hair detangled.

In some embodiments, the compositions herein can leave hair looking fuller.

In some embodiments, the compositions herein can leave hair looking thicker.

In some embodiments, the compositions herein can leave hair feeling more voluminous.

In some embodiments, the compositions herein can provide hair having more volume.

In some embodiments, the compositions herein can leave hair looking more vibrant.

In some embodiments, the compositions herein can leave hair feeling silky.

Consumer testimonials, comments, and claims regarding the compositions of the present disclosure have been collected. Regarding the shampoo and conditioner compositions described herein, a third party consumer study with 83 subjects was conducted for 14 days. The results for the shampoo composition are shown below in Table 1. The results for the conditioner composition are shown below in Table 2. Regarding the milk serum composition described herein, a third party consumer study with 81 subjects was conducted and results are shown below in Table 3. No adverse effects were reported in any of the studies below.

TABLE 1
Shampoo consumer claims (Day 14)

| | % Strongly Agree & |
|---|---|
| Claims | Agree |
| Thoroughly cleanses | 93 |
| Did not fade my hair color | 93 (n = 54) |
| I can wear a black shirt after using this | 92 |
| product (does not cause dandruff) | |
| Rinses out easily | 92 |
| Hair feels clean | 90 |
| Leaves no residue | 90 |
| Scalp does not feel dry or itchy | 90 |
| Hair feels smooth | 90 |
| Like the lather of the product | 90 |
| Scalp feels clean | 89 |
| Thoroughly lathers | 89 |
| Like the texture & consistency of the product | 89 |
| Hair looks healthy | 88 |
| Hair feels hydrated | 87 |
| Hair feels healthy | 86 |
| Did not strip hair | 86 |
| Hair feels free of buildup | 86 |
| Scalp feels healthy | 86 |
| My hair feels soft to the touch | 86 |
| like the scent of the product | 84 |
| Hair feels soft | 84 |
| Scalp feels balanced | 82 |
| Hair is noticeably softer | 82 |
| My scalp feels fresh | 80 |
| Enhances natural healthy texture | 80 |

TABLE 2
Conditioner consumer claims (Day 14)

| | % Strongly Agree & |
|---|---|
| Claims | Agree |
| I can wear a black shirt after using this | 94 |
| product (does not cause dandruff) | |
| Hair feels conditioned | 86 |
| Did not fade my hair color | 85 (n = 54) |
| Hair feels healthy | 84 |
| My hair feels instantly soft | 84 |
| My hair feels nourished | 84 |
| Rinses out easily | 84 |
| Like the scent of the product | 83 |
| My hair feels soft to the touch | 83 |
| Hair looks shiny | 82 |
| Hair is detangled | 81 |
| Easy to detangle hair after using this product | 80 |
| Hair appears less damaged | 80 |

TABLE 3
Milk Serum consumer claims

| Claims | % Strongly Agree & Agree |
|---|---|
| Like the texture & consistency of the product | 100 |
| Hair feels soft | 96 |
| Does not leave hair feeling crunchy | 95 |
| Did not fade hair color | 94 (n = 53) |
| Hair feels smooth and soft to the touch | 94 |
| My hair feels soft to the touch | 94 |
| Feels lightweight on hair | 93 |
| Hair feels hydrated | 91 |
| Leaves hair feeling moisturized | 91 |
| Feels lightweight in my hair | 91 |
| Like the scent of this product | 91 |
| Hair looks hydrated | 89 |
| My hair looks less frizzy | 89 |
| Easily tames frizz and fly-aways | 88 |
| Gives hair a smooth, defined appearance | 88 |
| Hair looks smoother | 88 |
| Provides frizz control | 88 |
| My hair is more manageable | 86 |
| Compared to other styling products I've | 86 |
| used, this product feels lighter- weight | |
| Hair looks healthier | 85 |
| Easily defrizzes my hair | 85 |
| Feels buildable and layerable | 85 |
| This product has made styling easier | 84 |
| Hair appears less damaged | 83 |
| Provides long lasting frizz control | 81 |
| Hair is detangled | 80 |
| Detangles hair | 80 |

As can be seen from the Tables above, most survey subjects reported positive opinions about the compositions described herein.

### EXAMPLES

### Example 1. Composition Formulation

Ginger extract, grape extract, frankincense, and walnut extract are emulsified in water and bees wax is added as a thickener. Citrus oil and citric acid are added. The composition is stored in an airtight container.

### Example 2. Evaluation of the Microscopic Condition of Hair Treated with a Mask Formulation Comprising the Composition of Example 1

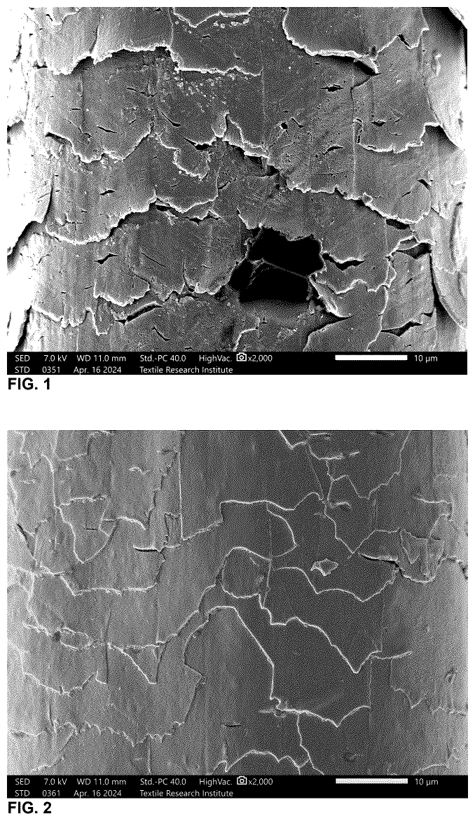

European Medium Brown hair fibers, 9% hydrogen peroxide bleached and fatigued for 3000 cycles to induce damage, were used in a hair tress treatment group and a hair tress control group. 3 fibers per cell, imaged at 3 locations per fiber, were used in the evaluation. All treatment was performed with an intellifaucet set at 40° C. with controlled flow rate of 1 GPM. Cell 1 had no treatment of the hair tresses, while the hair tresses of cell 2 were treated with a mask formulation comprising the composition of Example 1. Treatment was as follows: on damp hair, mask at 20% by weight of the tress (0.2 ml/g of hair) was applied, massaged 30 seconds, left on 5 minutes, rinsed 30 sec. Three hair fibers were randomly selected from the surface of the tress per treatment group. The hair fibers were attached to the surface of a 2.5 cm diameter stainless steel disc using double-sided copper tape and coated with Platinum to approximately 7 nm. The Scanning Electron Microcopy (SEM) pictures were recorded at 7 KV with a working distance of ˜11 mm at 600× and 2000× magnification. Analysis of the images obtained by SEM (using an JEOL JSM-IT200LV instrument) show that the mask formulation smoothed lifted cuticle edges in comparison with untreated control fibers (see FIG. 1 for control fiber and FIG. 2 for treated fiber). The surface condition of the damaged fibers improved after treatment with the mask formulation.

### Example 3. Evaluation of the Change in Breakage Properties of Hair Via Repeated Grooming when Treated with a Mask Formulation Comprising the Composition of Example 1

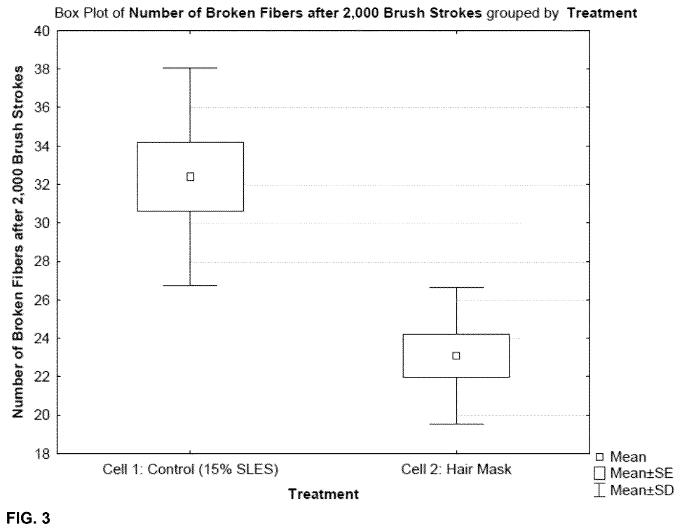

Ten European Medium Brown tresses were bleached using a 9% hydrogen peroxide solution at pH of 10.2. The bleached hair tresses, (3.0 g, 8″ length, 1″ wide) were used in a treatment group and a control group. All treatment was performed with an intellifaucet set at 40° C. with controlled flow rate of 1 GPM. All tresses were standardized with 15% sodium laureth sulfate (SLES) prior to treatments. For the control group, on damp hair, 15% SLES at 10% by weight of the tress (0.1 ml/g of hair) was applied, massaged 30 sec, rinsed 30 sec. For the treatment group, on damp hair, mask at 20% by weight of the tress (0.2 ml/g of hair) was applied, massaged 30 seconds, left on 5 minutes, rinsed 30 sec. All tresses were allowed to equilibrate at ambient temperature and 60% RH. After tresses were treated as outlined above, they were groomed using an automatic comber for a total of 2,000 cycles (˜50 strokes/minute). Fibers are collected and assessed after every 200 cycles. This methodology was adapted from Evans & Park, “A statistical analysis of hair breakage. II. Repeated grooming experiments.” Journal of Cosmetic Science 61 (6) (2010): 439-455. Box and whisker plots were generated using Statistica™, while JMP™ analytical software was used to calculate the statistics (student's t-test at 95% confidence level). Results from repeated grooming testing at 2,000 brush strokes are shown in FIG. 3. When compared to the control group, the treatment group was statistically different in breakage after 2,000 brush strokes. The treatment group measured an approximate 31% decrease in breakage when compared with the control (see FIG. 3).

### Example 4. Evaluation of Anti-Static Properties

European Medium Brown hair fibers are bleached using 9% hydrogen peroxide and fatigued for 3000 cycles to induce damage, and are used in a hair tress treatment group and a hair tress control group. 3 fibers per cell, imaged at 3 locations per fiber, are used in the evaluation. All treatment is performed with an intellifaucet set at 40° C. with controlled flow rate of 1 GPM. Cell 1 has no treatment of the hair tresses, while the hair tresses of cell 2 are treated with a mask or a milk serum formulation comprising the composition of Example 1. Treating is as follows: on damp hair, apply mask (or milk serum) at 20% by weight of the tress (0.2 ml/g of hair), massage 30 seconds, leave on 5 minutes, rinse 30 sec. Three hair fibers are randomly selected from the surface of the tress per group. The hair fibers with treatment show less electrostatic forces than the control fibers using static electricity testing.

### Example 5. Evaluation of Disulfide Bond Repair

The procedure of Example 4 is used to induce damage in a hair tress treatment group and a hair tress control group. The treatment group is treated with the mask or the milk serum. The hair fibers are chemically tested and show the testing group has more disulfide bonds than the control group.

### Example 6. Evaluation of Hair Health Based on Seasonal Changes

The procedure of Example 4 is used to induce damage in a hair tress treatment group and a hair tress control group at different relative humidities (RH). The treatment group is treated with the shampoo, conditioner, or mask. The RH in the experiments is 20%, 30%, 40%, 50%, 60% and 70%. The treatment group shows less breakage when compared with the control group.

### Example 7. Evaluation of Follicle Detoxification

A test using the shampoo or the hair scrub is used and shows the level of toxic metals in the scalp of the treatment group is less than the control group. This test is repeated at the RH's of Example 6, and shows the level of toxic metals in the scalp of the treatment group is less than the control group.

### Example 8. Evaluation of Hair Detoxification

The procedure of Example 4 is used to induce damage in a hair tress treatment group and a hair tress control group. The treatment group is treated with the shampoo or the hair scrub. The level of toxic metals in the hair of the treatment group is less than the control group.

### Example 9. Evaluation of Hair Growth Cycles

Hair growth cycles, specifically anagen, catagen, and telogen are evaluated over a period of time in a test group using the shampoo and the conditioner as described herein and a control group. At time intervals of 0 months, 1 month, 2 months, and 3 months, a small scalp area is shaved, and hair regrowth is evaluated. A photo is taken and hair follicles are counted to test the anagen, catagen, and telogen at each time point. The test group had more hair growth than the control group.

### Example 10. Evaluation of Barrier Protection of Hair

The procedure of Example 4 is used to induce damage in a hair tress treatment group and a hair tress control group. The treatment group is treated with the shampoo, the conditioner, the milk serum, or the mask. TEWL and FTIR spectroscopy are used in evaluation of the groups. The treatment group shows greater hydrophobicity when compared with the control group. In a study using sunlight, the treatment group shows more melanin protection than the control group.

### Example 11. Evaluation of Barrier Protection of Scalp

A test evaluates the scalp over a period of time in a test group using the shampoo, the conditioner, the milk serum, or the mask as described herein and a control group. At time intervals of 0 months, 1 month, 2 months, and 3 months, a small scalp area is shaved, and is evaluated using TEWL and FTIR spectroscopy on the sample. The treatment group shows greater hydrophobicity when compared with the control group. In a study using sunlight, the treatment group shows more melanin protection than the control group.

### Example 12. Evaluation of Hair Follicle DNA Protection

The scalp samples of Example 11 are used to evaluate DNA protection against UV exposure and oxidative stress. The treatment group shows less DNA damage and less oxidative stress when compared with the control group.

### Example 13. Evaluation of Color Protection and Oxidation

The procedure of Example 4 is used to induce damage in a hair tress treatment group and a hair tress control group. The treatment group is treated with the shampoo, conditioner, hair mask, or milk serum. The treatment group shows more color protection and less oxidation and more disulfide bonds when compared with the control group.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

I/We claim:

1. A composition, wherein
the composition is primarily formulated of natural ingredients, and
wherein the composition is configured to be administered to the hair, scalp and/or skin without any substantial damage to the hair, scalp, and/or skin.

2. The composition of claim 1, comprising ginger root extract, grape extract, walnut extract, and frankincense.

3. The composition of claim 2, further comprising at least one of the following additives: a thickener, a surfactant, a dye, a fragrance, and/or a preservative.

4. The composition of claim 2, wherein the composition is in a form of a shampoo, a dry shampoo, a conditioner, a dry conditioner, a lotion, a cream, a spray, a powder, a serum, a milk serum, a gel, a mask, a spray, a mist, a balm, a foam, a wax, an exfoliant, a scrub, or a combination of forms.

5. The composition of claim 2, wherein the composition imparts a silky wet feel to hair and inhibits fly-away up to about 35% relative humidity.

6. The composition of claim 2, wherein the composition is rinsed off after application to the hair and/or scalp.

7. The composition of claim 2, wherein the composition is applied to the hair and/or scalp and is not rinsed off.

8. The composition of claim 2, wherein the composition has substantially reduced product build up after repeated use.

9. The composition of claim 2, wherein the composition has a pH in the range of about 4.5 to about 7.

10. The composition of claim 2, wherein the composition has a pH of about 7.

11. The composition of claim 2, wherein the composition retains hair color and reduces breakage of hair strands.

12. The composition of claim 2, wherein the composition is a hair and scalp conditioning composition.

13. The composition of claim 2, comprising about 0.01% to about 3% of ginger root extract by weight.

14. The composition of claim 2, comprising about 0.01% to about 3% of grape extract by weight.

15. The composition of claim 2, comprising about 0.01% to about 3% of walnut extract by weight.

16. The composition of claim 2, comprising about 0.01% to about 3% of frankincense by weight.

17. The composition of claim 2, wherein the combination of ginger root extract, grape extract, walnut extract, and frankincense comprise about 0.5% to about 5% of the total weight of the composition.

18. The composition of claim 2, wherein the combination of ginger root extract, grape extract, walnut extract, and frankincense comprise about 2% of the total weight of the composition.

19. The composition of claim 2, wherein the composition is applied on a daily basis.

20. A method of treating hair, skin, or a scalp, comprising administering a composition primarily formulated of natural ingredients to a subject in need thereof, wherein the composition is a shampoo, a dry shampoo, a conditioner, a dry conditioner, a lotion, a cream, a spray, a powder, a serum, a milk serum, a gel, a mask, or a combination of forms.
